(19) United States
(12) Patent Application Publication
(10) Pub. No.: US 2011/0070199 A1
(43) Pub. Date: Mar. 24, 2011

(54) ADAPTABLE MODIFIED VIRUS VECTOR TO DELIVER RIBOSOMAL RIBONUCLEIC ACID COMBINED WITH MESSENGER RIBONUCLEIC ACID AS A MEDICAL TREATMENT DEVICE TO MANAGE DIABETES MELLITUS AND OTHER PROTEIN DEFICIENT DIEASES

(76) Inventors: Lane Bernard Scheiber, II, Gross Ile, MI (US); Lane Bernard Scheiber, Annandale, VA (US)

(21) Appl. No.: 12/564,022

(22) Filed: Sep. 21, 2009

(51) Int. Cl. A61K 35/76; A61K 31/7088

(52) U.S. Cl. 424/93.6; 514/44 R

(57) ABSTRACT

Diabetes mellitus is a disease of elevated blood glucose, often directly related to a deficiency in insulin production or insulin receptor production. The innovative strategy of treatment described here utilizes modified viruses and virus-like vehicles to act as a transport mechanism to deliver ribosomal RNA molecules along with messenger RNA molecules to target cells in the body. Delivering to the Beta cells in the body the ribosomal RNA needed to assist ribosomes with the construction of insulin or insulin receptors along with messenger RNA will lead to enhanced production of biologically active insulin or insulin receptors by Beta cells as necessary, which will lead to correcting deficiencies in insulin or insulin receptors the result of which will help properly regulate blood glucose levels throughout the body utilizing innate regulatory mechanisms.

### CROSS-REFERENCE TO RELATED APPLICATIONS

None.

### STATEMENT REGARDING SPONSORED RESEARCH OR DEVELOPMENT

None.

### REFERENCE TO SEQUENCE LISTING, A TABLE, OR COMPUTER LISTING COMPACT DISC APPENDIX

Not applicable.

©2009 Lane B. Scheiber II and Lane B. Scheiber. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to correct a protein deficiency in the body by increasing the intracellular production of the deficient protein by utilizing a modified virus to insert one or more ribosomal ribonucleic acid molecules along with one or more messenger ribonucleic acid molecules into one or more cells of the body.

2. Description of Background Art

For purposes of this text there are several general definitions. A ‘ribose’ is a five carbon or pentose sugar (C₅H₁₀O₅) present in the structural components of ribonucleic acid, riboflavin, and other nucleotides and nucleosides. A ‘deoxyribose’ is a deoxypentose (C₅H₁₀O₄) found in deoxyribonucleic acid. A ‘nucleoside’ is a compound of a sugar usually ribose or deoxyribose with a nitrogenous base by way of an N-glycosyl link. A ‘nucleotide’ is a single unit of a nucleic acid, composed of a five carbon sugar (either a ribose or a deoxyribose), a nitrogenous base and a phosphate group. There are two families of ‘nitrogenous bases’, which include: pyrimidine and purine. A ‘pyrimidine’ is a six member ring made up of carbon and nitrogen atoms; the members of the pyrimidine family include: cytosine (C), thymine (T) and uracil (U). A ‘purine’ is a five-member ring fused to a pyrimidine type ring; the members of the purine family include: adenine (A) and guanine (G). A ‘nucleic acid’ is a polynucleotide which is a biologic molecule such as ribonucleic acid or deoxyribonucleic acid that allow organisms to reproduce. A ‘ribonucleic acid’ (RNA) is a linear polymer of nucleotides formed by repeated riboses linked by phosphodiester bonds between the 3-hydroxyl group of one and the 5-hydroxyl group of the next; RNAs are a single strand macromolecule comprised of a sequence of nucleotides, these nucleotides generally referred to by their nitrogenous bases, which include: adenine, cytosine, guanine or uracil. RNAs are subset into different types which include messenger RNA (mRNA), transport RNA (tRNA), and ribosomal RNA (rRNA). Messenger RNAs act as templates to produce proteins. A ribosome is a complex comprised of rRNAs and proteins and is responsible for the correct positioning of mRNA and charged tRNA to facilitate the proper alignment and bonding of amino acids into a strand to produce a protein. A ‘charged’ tRNA is a tRNA that is carrying an amino acid. Ribosomal RNA (rRNA) represents a subset of RNAs that form part of the physical structure of a ribosome.

Diabetes mellitus represents an important health issue that affects a significant portion of the world population. In the United States, about 16 million people suffer from diabetes mellitus. Every year, about 650,000 additional people are diagnosed with this disease. Diabetes mellitus is the seventh leading cause of all deaths.

Diabetes mellitus represents a state of hyperglycemia, a serum blood sugar that is higher than what is considered the normal range for humans. Glucose, a six-carbon molecule, is a form of sugar. Glucose is absorbed by the cells of the body and converted to energy by the processes of glycolysis, the Krebs cycle and phosporylation. Insulin, a protein, facilitates the transfer of glucose from the blood into cells. Normal range for blood glucose in humans is generally defined as a fasting blood plasma glucose level of between 70 to 110 mg/dl. For descriptive purposes, the term ‘plasma’ refers to the fluid portion of blood.

Diabetes mellitus is classified as Type One and Type Two. Type One diabetes mellitus is insulin dependent, which refers to the condition where there is a lack of sufficient insulin circulating in the blood stream and insulin must be provided to the body in order to properly regulate the blood glucose level. When insulin is required to regulate the blood glucose level in the body, this condition is often referred to as insulin dependent diabetes mellitus (IDDM). Type Two diabetes mellitus is noninsulin dependent, often referred to as noninsulin dependent diabetes mellitus (NIDDM), meaning the blood glucose level can be managed without insulin, and instead by means of diet, exercise or intervention with oral medications. Type Two diabetes mellitus is considered a progressive disease, the underlying pathogenic mechanisms including pancreatic Beta cell (also often designated as β-Cell) dysfunction and insulin resistance.

The pancreas serves as an endocrine gland and an exocrine gland. Functioning as an endocrine gland the pancreas produces and secretes hormones including insulin and glucagon. Insulin acts to reduce levels of glucose circulating in the blood. Beta cells secrete insulin into the blood when a higher than normal level of glucose is detected in the serum. For purposes of this description the terms ‘blood’, ‘blood stream’ and ‘serum’ refer to the same substance. Glucagon acts to stimulate an increase in glucose circulating in the blood. Beta cells in the pancreas secrete glucagon when a low level of glucose is detected in the serum.

Glucose enters the body and then the blood stream as a result of the digestion of food. The Beta cells of the Islets of Langerhans continuously sense the level of glucose in the blood and respond to elevated levels of blood glucose by secreting insulin into the blood. Beta cells produce the protein ‘insulin’ in their endoplasmic reticulum and store the insulin in vacuoles until it is needed. When Beta cells detect an increase in the glucose level in the blood, Beta cells release insulin into the blood from the described storage vacuoles.

Insulin is a protein. An insulin protein consists of two chains of amino acids, an alpha chain and a beta chain, linked by two disulfide (S—S) bridges. One chain, the alpha chain consists of 21 amino acids. The second chain the beta chain consists of 30 amino acids.

Insulin interacts with the cells of the body by means of a cell-surface receptor termed the ‘insulin receptor’ located on the exterior of a cell's ‘outer membrane’, otherwise known as the ‘plasma membrane’. Insulin interacts with muscle and liver cells by means of the insulin receptor to rapidly remove excess blood sugar when the glucose level in the blood is higher than the upper limit of the normal physiologic range. Recognized functions of insulin include stimulating cells to take up glucose from the blood and convert it to glycogen to facilitate the cells in the body to utilize glucose to generate biochemically usable energy, and to stimulate fat cells to take up glucose and synthesize fat.

Diabetes Mellitus may be the result of one or more factors. Causes of diabetes mellitus may include: (1) mutation of the insulin gene itself causing miscoding, which results in the production of ineffective insulin molecules; (2) mutations to genes that code for the ‘transcription factors’ needed for transcription of the insulin gene in the deoxyribonucleic acid (DNA) to create messenger ribonucleic acid (mRNA) molecules, which facilitate the manufacture of the insulin molecule; (3) mutations of the gene encoding for the insulin receptor, which produces inactive or an insufficient number of insulin receptors; (4) mutation to the gene encoding for glucokinase, the enzyme that phosphorylates glucose in the first step of glycolysis; (5) mutations to the genes encoding portions of the potassium channels in the plasma membrane of the Beta cells, preventing proper closure of the channel, thus blocking insulin release; (6) mutations to mitochondrial genes that as a result, decreases the energy available to be used facilitate the release of insulin, therefore reducing insulin secretion; (7) failure of glucose transporters to properly permit the facilitated diffusion of glucose from plasma into the cells of the body.

A ‘eukaryote’ refers to a nucleated cell. Eukaryotes comprise nearly all animal and plant cells. A human eukaryote or nucleated cell is comprised of an exterior lipid bilayer plasma membrane, cytoplasm, a nucleus, and organelles. The exterior plasma membrane defines the perimeter of the cell, regulates the flow of nutrients, water and regulating molecules in and out of the cell, and has embedded into its structure receptors that the cell uses to detect properties of the environment surrounding the cell membrane. The cytoplasm acts as a filling medium inside the boundaries of the plasma cell membrane and is comprised mainly of water and nutrients such as amino acids, oxygen, and glucose. The nucleus, organelles, and ribosomes are suspended in the cytoplasm. The nucleus contains the majority of the cell's genetic information in the form of double stranded deoxyribonucleic acid (DNA). Organelles generally carry out specialized functions for the cell and include such structures as the mitochondria, the endoplasmic reticulum, storage vacuoles, lysosomes and Golgi complex (sometimes referred to as a Golgi apparatus). Floating in the cytoplasm, but also located in the endoplasmic reticulum and mitochondria are ribosomes. Ribosomes are complex macromolecule structures comprised of ribosomal ribonucleic acid (rRNA) molecules and ribosomal proteins that combine and couple to a messenger ribonucleic acid (mRNA) molecule. The rRNAs and the ribosomal proteins congregate to form a macromolecule structure that surrounds a mRNA molecule. Ribosomes decode genetic information in a mRNA molecule and manufacture proteins to the specifications of the instruction code physically present in the mRNA molecule. More than one ribosome may be attached to a single mRNA at a time.

The majority of the deoxyribonucleic acid (DNA) comprises the chromosomes, double stranded helical structures located in the nucleus of the cell. DNA in a circular form, can also be found in the mitochondria, the powerhouse of the cell, an organelle that assists in converting glucose into usable energy molecules. DNA represents the genetic information a cell needs to manufacture the materials it requires to sustain life and to replicate. Genetic information is stored in the DNA by arrangements of four nucleotides referred to as: adenine, thymine, guanine and cytosine. DNA represents instruction coding, that in the process known as transcription, the DNA's genetic information is decoded by transcription protein complexes referred to as polymerases (or polymerase complex), to produce ribonucleic acid (RNA). RNA is a single strand of genetic information comprised of coded arrangements of four nucleotides: adenine, uracil, guanine and cytosine. In a RNA, ‘uracil’ takes the place of ‘thymine’, thymine being present in the DNA. Several different types of RNAs have been identified, which include messenger RNAs (mRNA), transport RNAs (tRNA) and ribosomal RNAs (rRNA).

Proteins are comprised of a series of amino acids bonded together in a linear strand, sometimes referred to as a chain; a protein may be further modified to be a structure comprised of one or more similar or differing strands of amino acids bonded together. Insulin is a protein structure comprised of two strands of amino acids; one strand comprised of 21 amino acids long and the second strand comprised of 30 amino acids, the two strands attached by two disulfide bridges. There are an estimated 30,000 different proteins the cells of the human body may manufacture. The human body is comprised of a wide variety of cells, many with specialized functions requiring unique combinations of proteins and protein structures such as glycoproteins (a protein combined with a carbohydrate) to accomplish the required task or tasks a specialized cell is designed to perform. Forms of glycoproteins are known to be utilized as cell-surface receptors. Messenger RNAs (mRNA) are created by transcription of DNA, they generally migrate to other locations inside the cell and are utilized by ribosomes as protein manufacturing templates. A ribosome is a protein complex that manufactures proteins by deciphering the instruction code located in a mRNA molecule. When a specific protein is needed, pieces of the ribosome complex, which include rRNA molecules and ribosomal proteins, bind around the strand of a mRNA that carries the specific instruction code that will generate the required protein. The ribosome traverses the mRNA strand and deciphers the genetic information coded into the sequence of nucleotides that comprise the mRNA molecule to produce a protein molecule and this process is referred to as translation.

A ribosome is complex macromolecule comprised of ribosomal RNA (rRNA) molecules and ribosomal proteins. Ribosomal RNAs and ribosomal proteins are often designated with a number measured in Svedberg (S) units, which represents the sediment coefficient. The sediment coefficient is influenced by both molecular weight of the molecule and surface area of the molecule. In humans there are generally recognized at this time two mitochondrial rRNAs identified as 12S rRNA and 16S rRNA, and there are generally recognized four rRNAs that reside in the cytoplasm of a cell identified as 5S rRNA, 5.8S rRNA, 18S rRNA and 28S rRNA. There may be other rRNAs, as of yet unidentified, that reside in some of the other structures in the cell that engage in manufacturing of macromolecules, such as the smooth endoplasmic reticulum, the rough endoplasmic reticulum and the Golgi complex.

In eukaryotes, in the cytoplasm, the ribosome complex is referred to as an 80S ribosome. Generally two ribosomal proteins comprise a ribosome complex. This 80S ribosome is comprised of one ‘dome-shape’ 60S ribosomal protein and one ‘cap-shaped’ 40S ribosomal protein.

In the forms of life referred to as vertebrates (Humans are classified as a form of vertebrate), of the four rRNAs that reside in the cytoplasm of a eukaryote cell, the 18S rRNA is found in and helps comprise the physical structure of the 40S protein subunit of the ribosome complex and the 5S rRNA, 5.8S rRNA, and 28S rRNA molecules are found in and help comprise the physical structure of the 60S protein subunit of the ribosome complex.

The rRNA molecules are thought to provide at least three different functions for the ribosome complex. The rRNA molecules are thought to: (1) assist with identification of the messenger RNA to be translated, (2) act as an enzyme to facilitate the production of the protein molecule being translated from the mRNA molecule undergoing translation, (3) possibly cause folding at certain locations in the three dimensional structure of the protein being generated as the ribosome complex decodes the mRNA molecule.

Ribosomal RNAs (rRNA) are generated by polymerase molecules deciphering the instruction code present in the DNA. The rRNAs generally migrate to locations where mRNAs are to be utilized as templates. The rRNA molecules connect to their respective ribosome proteins and this macromolecule complex, referred to as a ribosome or ribosome complex, surrounds the beginning segment of a mRNA molecule. Utilizing inherent coding, the rRNA molecules direct the ribosome pieces to build the ribosome complex around a particular strand of mRNA or particular type of mRNA. Given there are four unique types of nucleotides that make up the physical linear strand of any RNA molecule, these four unique types of nucleotides may in some circumstances represent a base-four numbering system. As an analogy, at the core of the current digital computer technology, binary coding or base-two coding, which is comprised of ‘ones’ and ‘zeros’, is utilized in certain formats to code for names and numbers. The inherent coding the rRNA molecules harbor is a sequence of nucleotides which represent a unique ‘name’ (coded in the base-four numbering system), unique ‘base-four number’ or unique ‘combination of a name and base-four number’ that corresponds to a particular mRNA or particular type of mRNA. In this manner, rRNAs act to control which mRNA molecule will undergo translation to produce proteins, rather than a ribosome complex randomly engaging any mRNA template that happens to be available. With the DNA producing rRNA molecules that cause a ribosome to attach to a particular mRNA molecule or particular type of mRNA molecule, the DNA is able to exert control over the manufacturing capacity of the cell and produce proteins as needed, rather than producing proteins in a random fashion. Producing proteins as needed by the cell, rather than in a random fashion, conserves valuable resources and conserves energy inside the cell.

RNAs are generally degraded by enzymes known as ribonucleases or RNAases. Ribonucleases act to split RNA molecules, which tends to inactive the RNA molecules; preventing RNA molecules from performing their duties. Different RNAs have different half-lives. A ‘service life’ refers to the amount of time an RNA molecule may participate in the biologic functions it was originally created to participate in within a cell. A ‘half-life’ or ‘service half-life’ is the amount of time it takes for half of a given amount of RNA molecules to degrade to a form to which the RNA molecules are unable to participate in the biologic processes they were created to participate in within a cell. The ‘life’ or ‘service life’ or ‘time span of participation in biologic processes’ of biologic macromolecules is generally measured and reported in the science community in terms of the macromolecule's half-life. The nucleotide sequencing of the rRNA molecule could be altered from that of the naturally occurring molecule to lengthen or shorten the service half-life of the native rRNA. Lengthening the service life of rRNA molecules such that the rRNAs could combine with ribosomal proteins to participate in ribosomes over a longer than the naturally occurring period of time would be especially useful in diabetic patients to facilitate a greater production of insulin molecules in the Beta cells.

The rate of degradation of rRNA molecules by ribonucleases could be varied by changing the nucleotide sequence of the rRNA molecule or by altering the folding characteristics of the rRNA molecule or by altering both the nucleotide sequence and the folding characteristics of the rRNA molecule. By making changes to the nucleotide sequence and/or the folding characteristics of the rRNA molecules the rate of degradation of the rRNA molecules by ribonucleases could be caused to vary in length of time from seconds to minutes to hours to days to weeks to months to years.

Transport RNAs (tRNA) are constructed in the nucleus or in the mitochondria, and are coded for one of the 20 amino acids the cells of the human body use to construct proteins. Once a tRNA is created by transcription of the DNA, the tRNA seeks out the type of amino acid it has been coded for and attaches to that specific amino acid. A tRNA molecule is considered to be ‘charged’ when it is carrying an amino acid. The charged tRNA delivers the amino acid it carries to a ribosome that is waiting for the specific amino acid the tRNA is carrying. Proteins are manufactured by the ribosomes binding together sequences of amino acids. The order by which the amino acids are bonded together is dictated by the way the sequencing of the nucleotides comprising the mRNA is constructed and how the ribosome interprets the information encoded in the string of nucleotides present in the mRNA strand.

Messenger RNA molecules are divided into three regions. The three regions include the (1) 5′ untranslatable region, (2) the coding region, and (3) the 3′ untranslatable region. An ‘untranslatable region’ represents a segment of a messenger RNA molecule that does not code for a protein and is not used to yield a protein and therefore ‘translation’ does not occur in such a region. The ‘coding region’ is the portion of the mRNA that is decoded by the ribosomes by the process known as translation to produce a particular protein molecule. A sequence of three nucleotides present in the coding region of a mRNA molecule represents a unit of information referred to as a codon. Codons code for all of the 20 amino acids used to construct protein molecules and also for START and STOP commands. In the process known as translation, the ribosome decodes the codons present in the coding region in the mRNA, initiating the protein manufacturing process at a START codon, then interfacing with charged tRNAs carrying the amino acids that match the sequence of codons in the mRNA as the ribosome traverses the length of the coding region of the mRNA molecule. The ribosome functions as a protein factory by taking amino acids delivered by tRNAs and binding the amino acids together in the order dictated by the sequence of codon instructions coded into the mRNA template as directed by the manner of the nucleic acid arrangement in the mRNA molecule. Protein synthesis ceases when a ribosome encounters a STOP code. The protein molecule is then released by the ribosome. Ribosomes do not decode the nucleotide sequences to produce proteins in a mRNA's 5′ untranslatable region or a mRNA's 3′ untranslatable region.

The insulin molecule is a protein produced by Beta cells located in the pancreas. The ‘insulin messenger RNA’ is created in a Beta cell by a polymerase complex transcribing the insulin gene from nuclear DNA in the nucleus of the cell. The native messenger RNA (mRNA) for insulin then travels to the endoplasmic reticulum, where numerous ribosomes, comprised of rRNA and ribosomal proteins, engage these mRNA molecules. Many ribosomes may be attached to a single strand of mRNA simultaneously, each generating an identical copy of the protein as dictated by the information encoded in the mRNA. Insulin is produced by ribosomes translating the information in a mRNA molecule coded for the insulin protein, which produce strands of amino acids that are coded for an immature form of the biologically active insulin molecule referred to as ‘pro-insulin’. Once the pro-insulin molecule is generated it then undergoes modification by several enzymes including prohormone convertase one (PC1), prohormone convertase two (PC2) and carboxypeptidase E, which results in the production of a biologically active insulin molecule. Once the biologically active insulin protein is generated it is stored in a vacuole in the Beta cell to await being released into the blood stream.

Insulin receptors, which appear on the surface of cells, offer binding sites for insulin circulating in the blood. When insulin binds to an insulin receptor, the biologic response inside the cell causes glucose to enter the cell and undergo processing in the cytoplasm. Processed glucose molecules then enter the mitochondria. The mitochondria further process the modified glucose molecules to produce usable energy in the form of adenosine triphosphate molecules (ATP). Thirty-eight ATP molecules may be generated from one molecule of glucose during the process of aerobic respiration. ATP molecules are utilized as an energy source by biologic processes throughout the cell.

The current medical therapeutic approach to the management of diabetes mellitus has produced limited results. Patients with diabetes generally struggle with an inadequate production of insulin, or an ineffective release of biologically active insulin molecules, or a release of an insufficient number of biologically active insulin molecules, or an insufficient production of cell-surface receptors, or a production of ineffective cell-surface receptors, or a production of ineffective insulin molecules that are unable to interact properly with insulin receptors to produce the required biologic effect. Type One diabetes requires administration of exogenous insulin. The traditional approach to Type Two diabetes has generally first been to adjust the diet to limit the caloric intake the individual consumes. Exercise is used as an initial approach to both Type One and Type Two diabetes as a means of up-regulating the utilization of fats and sugar so as to reduce the amount of circulating plasma glucose. When diet and exercise are inadequate in properly managing Type Two diabetes, oral medications are often introduced. The action of sulfonylureas, a commonly prescribed class of oral medication, is to stimulate the Beta cells to produce additional insulin receptors and enhance the insulin receptors' response to insulin. Biguanides, another form of oral treatment, inhibit gluconeogenesis, the production of glucose in the liver, thereby attempting to reduce plasma glucose levels. Thiazolidinediones (TZDs) lower blood sugar levels by activating peroxisome proliferator-activated receptor gamma (PPAR-γ), a transcription factor, which when activated regulates the activity of various target genes, particularly ones involved in glucose and lipid metabolism. If diet, exercise and oral medications do not produce a satisfactory control of the level of blood glucose in a diabetic patient, exogenous insulin is injected into the body in an effort to normalize the amount of glucose present in the serum. Insulin, a protein, has not successfully been made available as an oral medication to date due to the fact that proteins in general become degraded when they encounter the acid environment present in the stomach.

Despite strict monitoring of blood glucose and potentially multiple doses of insulin injected throughout the day, many patients with diabetes mellitus still experience devastating adverse effects from elevated blood glucose levels. Microvascular damage and elevated tissue sugar levels contribute to such complications as renal failure, retinopathy involving the eyes, neuropathy, and accelerated heart disease despite aggressive efforts to maintain the blood sugar within the physiologic normal range using exogenous insulin by itself or a combination of exogenous insulin and one or more oral medications. Diabetes remains the number one cause of renal failure in the United States. Especially in diabetic patients that are dependent upon administering exogenous insulin into their body, though dosing of the insulin may be four or more times a day and even though this may produce adequate control of the blood glucose level to prevent the clinical symptoms of hyperglycemia; this does not unerringly supplement the body's natural capacity to monitor the blood sugar level minute to minute, twenty-four hours a day, and deliver an immediate response to a rise in blood glucose by the release of insulin from Beta cells as required. The deleterious effects of diabetes may still evolve despite strict and persistent control of the glucose level in the blood stream.

The current treatment of diabetes may be augmented by the unique approach to utilizing modified viruses as vehicles to transport ribosomal ribonucleic acid (rRNA) molecules along with exogenous messenger ribonucleic acid (mRNA) into cells in order to increase the production of biologically active insulin. By utilizing modified viruses to transport rRNA to facilitate assembly of ribosomes that are intended to attach to exogenous messenger ribonucleic acid molecules delivered into the cell by the modified virus or to attach to native messenger ribonucleic acid (mRNA) molecules already present in the cell would offer a new treatment option for patients with diabetes. By delivering both rRNA along with mRNA coded to facilitate the manufacture of pro-insulin, insulin, the enzymes utilized to modify proinsulin to the biologically active insulin molecule and/or insulin receptors, the Beta cells in the pancreas would be stimulated to generate additional insulin. The above-mentioned concept would be analogous to providing cells with both the ‘elements of the decoder’ and the ‘template’ needed to construct copies of one or more proteins that a cell is lacking.

Viruses are obligate parasites. Viruses simply represent a carrier of genetic material and by themselves viruses are unable to replicate or carry out any form of biologic function outside their host cell. Viruses are generally comprised of one or more nested shells constructed of one or more layers of protein or lipid material, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's probes is to locate and engage a host cell's receptors. The virus's surface probes are designed to detect, make contact with and functionally engage one or more receptors located on the exterior of a cell type that will offer the virus the proper environment in which to construct copies of itself. A host cell provides the virus the proper biochemical machinery for the virus to successfully replicate itself.

Protected by an outer protein coat or lipid envelope, viruses carry a genetic payload in the form of deoxyribonucleic acid (DNA) or ribonucleic acid (RNA). Once a virus's exterior probes locate and functionally engage the surface receptor or receptors on a host cell, the virus inserts its genetic payload into the interior of the host cell. In the event a virus is carrying a DNA payload, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of their RNA by having the RNA converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA (mRNA). RNA viruses that bypass the host cell's DNA, cause the cell to in general generate copies of the necessary parts of the virus directly from the virus's RNA genome.

The Hepatitis C virus (HCV) is a positive sense RNA virus, meaning HCV's genome is a type of RNA that is capable of bypassing the need for involving the host cell's nucleus by having its RNA genome function as messenger RNA. The Hepatitis C virus infects liver cells. The Hepatitis C viral genome becomes divided once it gains access to the interior of a liver host cell. Portions of the subdivisions of the Hepatitis C viral genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein E1 probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its viral RNA genome payload through the plasma cell membrane of the liver cell, thus delivering the HCV RNA genome into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

A modified Hepatitis C virus offers a naturally occurring vehicle mechanism to transport and insert medically therapeutic ribosomal ribonucleic acid (rRNA) molecules along with one or more messenger ribonucleic acid (mRNA) molecules into specific targeted cells of the human body. The surface probes present on the Hepatitis C virus's outer protein coat can be modified to seek out specific receptors on specific target cells. Once the modified Hepatitis C virus's probes properly engage the cell-surface receptors on a target cell, the modified Hepatitis C virus would insert into the target cell one or more medically therapeutic ribosomal ribonucleic acid (rRNA) molecules along with one or more messenger ribonucleic acid (mRNA) molecules for the purpose of having the target cell generate proteins to achieve a medically therapeutic response.

Current state of gene therapy generally refers to efforts directed toward inserting an exogenous subunit of DNA into a vehicle such as a naturally occurring virus. The vehicle is intended to insert the exogenous subunit of DNA into a cell the virus naturally targets. The exogenous DNA subunit then migrates to the target cell's nucleus. The exogenous DNA subunit then inserts into the native DNA of the cell. This represents a permanent alteration of the cell's nuclear DNA. The nuclear transcription proteins then read the exogenous DNA subunit's nucleotide coding to produce the intended cellular response. The approach described hereunder involves RNA versus DNA. DNA is comprised of the nucleotides: adenine, thymine, guanine and cytosine. RNA is composed of the nucleotides: adenine, uracil, guanine and cytosine. DNA codes for the manufacture of RNAs, which are composed of nucleotides. RNAs facilitate the manufacture of proteins, which are composed of amino acids. The virus chosen as the transport vehicle, Hepatitis C virus, is a RNA virus versus a virus that naturally carries a DNA genome.

Beta cells located in the Islets of Langerhans in the pancreas are thought to have at least one unique identifying surface receptor. The exterior receptor GPR40 appears specific to Beta cells located in the Islets of Langerhans in the pancreas. A virus equipped with a surface probe designed to engage the GPR40 Beta cell receptor, could travel the blood stream of the body until it locates a GPR40 receptor on a Beta cell, engage the receptor with its surface probe, and then insert the genetic payload it carries into the Beta cell. A genetic payload of one or more ribosomal RNAs along with one or more messenger RNAs could be used to enhance proper protein production by cells deficient in a particular protein. Hormones are proteins that circulate the body and stimulate biologic activity specific to the hormone's role. In the case of a deficiency of a hormone, production of a deficient hormone could be enhanced by inserting one or more ribosomal RNAs along with one or more messenger RNAs into specific target cells in the body to stimulate production of the required hormone. In the case of diabetes mellitus, utilizing a modified Hepatitis C virus as a vehicle, ribosomal RNA molecules along with messenger RNA molecules could be inserted into Beta cells, to assist the Beta cells are with generating an adequate insulin production and adequate release of insulin into the blood to meet the body's needs.

Viruses are constructed in a number of ways and shapes. The shape of a virus may be rod or filament like, icosahedral, or complex structures combining filament and polygonal shapes. Viruses may have an outer protein coat or an outer envelope comprised of lipids. Icosahedral viruses with an outer lipid envelope appear spherical in shape. An outer envelope is often comprised of two lipid layers often termed a lipid bilayer. Spherical viruses may be in the form of being comprised of an outer envelope and one inner shell or an outer envelope and two inner shells. In the case of a spherical virus with an outer envelope and one inner shell, the inner shell is often referred to as a nucleocapsid shell comprised of capsid proteins. In the case of a spherical virus being comprised of an outer envelope and two inner shells, the outer most inner viral shell may be referred to as comprised of matrix proteins, the innermost of the two shells being referred to as a nucleocapsid shell being comprised of capsid proteins. The inner protein shell is nested inside the outer protein shell.

An alternative means to treat diabetes might include the use of a quantity of virus-like transport vehicles. The virus-like transport vehicles would in general be spherical in shape; though other shapes may be used as function might warrant the use of a particular shape. The spherical virus-like transport vehicles would be comprised of a lipid bilayer envelope and one or more inner nested protein shells, depending upon the size of the payload. Nesting of protein shells refers to progressively smaller diameter shells fitting snugly inside protein shells of a larger diameter. The virus-like transport vehicles would be similar in construction to viruses, and such virus-like transport vehicles would carry a payload consisting of ribosomal RNA molecules. The size of the virus-like transport vehicle would depend upon the payload the virus-like transport vehicle would be required to carry. Embedded in the virus-like transport vehicle's outer lipid bilayer surface would be a quantity of probes that are intended to target cell-surface receptors on specific cells. The virus-like transport vehicle would be introduced into a patient's blood stream or tissues so that the virus-like transport vehicle could deliver the therapeutic genetic payload that it carries to Beta cells in the pancreas. When one or more probes on the surface of the virus-like transport vehicle engage one or more cell-surface receptors on a Beta cell located in the pancreas, the virus-like transport vehicle will insert its therapeutic payload of ribosomal RNA into the Beta cell to enhance the Beta cell's biologic function of producing insulin and/or insulin receptors.

The action of altering the type of probe or probes present on the surface of the modified virus, the modified Hepatitis C virus, or the virus-like transport vehicle would change the target the modified virus or virus-like transport vehicle would seek out. By changing the probe on the surface of the modified virus, the modified Hepatitis C virus, or the virus-like transport vehicle the payload carried by the modified virus, the modified Hepatitis C virus, or the virus-like transport vehicle could be delivered to any cell that carried on its surface a cell-surface receptor that would engage the probes the modified virus, the modified Hepatitis C virus, or the virus-like transport vehicle was carrying on its surface. In this fashion, specific payloads could be delivered to specific cells throughout the body.

The utilization of ribosomal RNA molecules or messenger RNA molecules does not alter the cell's DNA. Ribosomal RNAs and messenger RNAs degrade due to the presence of RNAases and become unusable after a period of time. Use of RNA as a therapeutic modality offers a therapeutic opportunity that could have a reversible or an attenuable effect when required. Using ribosomal RNA and messenger RNA to produce proteins bypasses the action of decoding the DNA and errors or deficiencies that might occur during the process of transcription. By employing a medically therapeutic virus to carry ribosomal RNA along with messenger RNAs to cells, deficiencies of any of the approximately 30,000 proteins that comprise the tissues that exist in the body and on the surface of the body can be successfully treated or averted.

Messenger RNA molecules are comprised of three regions (or segments). These three regions include (1) a 5′ untranslatable region, (2) a coding region and (3) a 3′ untranslatable region. The ‘5′ untranslatable region’ acts as the initiation point for a ribosome to attach to the mRNA. The ‘coding region’ acts as the template from which a protein is constructed. An ‘untranslatable region’ represents a segment of a messenger RNA molecule that does not code for a protein and is not used to yield a protein and therefore ‘translation’ does not occur in such a region. The 3′ untranslatable region is associated with the degradation of the usefulness of the mRNA. Different mRNAs have different service life expectancies. The half-life of the naturally occurring mRNA that acts as the template responsible for the production of the protein ‘glucokinase’ is two hours. The half-life of the naturally occurring mRNA that acts as the template that produces the protein ‘alcohol dehydrogenase’ is ten hours. The half-life of the naturally occurring mRNA that acts as the template to produce the protein ‘glucuronidase’ is thirty hours. By modifying the nucleotides that comprise the 3′ untranslatable unit of an mRNA the service half-life of the mRNA may be altered to be lengthened or shortened depending upon the need for the quantity of protein and timeframe over which the mRNA is required to produce the protein coded in the protein template of the mRNA's coding region.

Research has demonstrated that natural proteins can be altered to produce medically beneficial effects. The parathyroid hormone (PTH) is one example. Intact PTH is produced by cells in the parathyroid glands. There are four parathyroid glands present in the neck, generally in the vicinity of the thyroid gland. The term ‘para-’ means ‘next to’, so early anatomists identified the four glands ‘parathyroid glands’ because they were generally found ‘next to’ the thyroid gland in the neck. Parathyroid hormone is released in response to the cells of the parathyroid gland sensing a decline in the level of serum calcium. Parathyroid hormone, in its natural state, acts to stimulate osteoclast cells present in bone to release calcium from bone, thereby returning the serum calcium level to the normal range. On the other hand, it has been quite well demonstrated that if (1) the amino acid chain of the parathyroid hormone is shortened and (2) the shorter parathyroid hormone molecule is pulsed, by injecting it into the body once a day, the action of this modified parathyroid hormone molecule is opposite of the intact parathyroid hormone. One such form of a shorter length parathyroid hormone molecule is termed ‘teriparatide’. Teriparatide (1-34) has the identical sequence from 1 to the 34^{th} N-terminal amino acid of the 84-amino acid endogenous human parathyroid hormone. The skeletal effects of the modified protein molecule act on bone cells to preferentially cause osteoblastic activity over osteoclastic activity, which results in storage of calcium into bone, rather than a release of calcium from bone if the teriparatide is administered once a day. Teriparatide has been a recognized and widely used treatment of osteoporosis since at least as far back as the year 2000.

Modifying the ‘coding region’ of a messenger RNA will modify the protein the messenger RNA will produce when the ribosomes decode such a modified messenger RNA. As demonstrated by the case of modifying the naturally occurring parathyroid hormone by administering a molecule that is comprised of fewer amino acids than the original PTH molecule, modifying proteins the messenger RNAs produce may provide health care providers with an entirely new and widely spanning armamentarium of medically beneficial therapies.

The 5′ untranslatable region of a messenger RNA molecule is used to identify the messenger RNA and utilized as a point of attachment by ribosomes to the messenger RNA molecule. Modifying the 5′ untranslatable region by altering the nucleotide sequence in the 5′ untranslatable region may make it easier to identify a modified messenger ribonucleic acid molecules in a fashion that the modified ribonucleic acid molecules can be engaged by ribosomes. Altering the nucleotide sequence of the 5′ untranslatable region of a modified messenger ribonucleic acid molecule to create a unique identifier would facilitate ribosomes to preferentially engage the modified messenger ribonucleic acid molecule to preferentially produce the protein for which the modified messenger ribonucleic acid molecule is acting as a template. Supplying cells with exogenous rRNA molecules and exogenous mRNA molecules that are both coded with a similar unique identifier such that the rRNA molecules will engage the mRNA molecules, facilitates the production of a desired protein.

A protein is a macromolecule consisting of long series of amino acids in a peptide linkage. A protease is any enzyme that catalyzes the hydrolysis of a protein in its initial stages of degradation to a simpler substance. Proteases either split proteins by removing amino acids from the ends of the protein or by splitting a protein into subunits. Proteases are divided into endopeptidases and exopeptidases. Exopeptidases catalyze the hydrolysis of the terminal amino acid of a protein chain. Exopeptidases include enzymes such as carboxypeptidase, aminopeptidases, dipeptidases. Endopeptidases catalyze the hydrolysis of a peptide chain well within the chain. Endopeptidases include enzymes such as pepsin, trypsin, cathepsins, papain.

A ‘messenger RNA’ carried by a modified form of virus, a modified Hepatitis C virus, or a virus-like transport vehicle may code or act as a template for ‘multiple proteins’. A messenger RNA may act as a template for multiple copies of the same protein. A messenger RNA may act as a template that will produce various different types of proteins. Viral genomes often code for multiple proteins on a single strand of genetic material. Viruses sometimes carry proteases, which are used to act on proteins generated by the viral genome, once the viral genome has been inserted inside a host cell. Utilizing naturally occurring proteases or exogenous proteases delivered with the messenger RNA or exogenous proteases delivered by a separate transport vehicle, the original protein produced by an exogenous messenger RNA molecule may be split into subunits, with each subunit representing a copy of the same protein or each subunit representing a different protein. By fitting multiple templates for a particular protein into a single exogenous messenger RNA and utilizing proteases to split the proteins generated by the exogenous messenger RNA at specific sites along the proteins, multiple copies of the same smaller protein subunit could be generated. By fitting multiple templates for different proteins into a single exogenous messenger RNA and utilizing proteases to split the proteins generated by this exogenous messenger RNA at specific sites along the proteins, one or more copies of different smaller protein subunits could be generated. The utilization of proteases may significantly increase the efficiency by which medically therapeutic proteins are generated inside a cell.

A messenger RNA carried by a modified form of virus, a modified Hepatitis C virus, or a virus-like transport vehicle may be comprised of multiple messenger RNA subunits. RNAs are generally degraded by enzymes known as ribonucleases or RNAases. Ribonucleases act to split RNA molecules, which generally represents a means to inactive the RNA molecules, except when ribonucleases are being used to divide the RNA molecule into smaller active components. Utilizing naturally occurring ribonucleases or exogenous ribonucleases delivered with the messenger RNA or exogenous ribonucleases delivered by a separate transport vehicle, the original exogenous messenger RNA molecule may be split into translatable subunits. The original exogenous messenger RNA molecule may be physically split or degraded into subunits, each subunit representing a separate template, that by the process of translation, will produce copies of the same protein or the original exogenous messenger RNA molecule may be physically split or degraded into subunits, each subunit representing a template, that by the process of translation, will produce copies of different proteins. The utilization of ribonucleases represents another technique that could significantly increase the efficiency by which medically therapeutic proteins are generated inside a cell.

### BRIEF SUMMARY OF THE INVENTION

A modified virus is used as a transport medium to carry a payload of one or more ribosomal ribonucleic acid molecules along with one or more messenger ribonucleic acid molecules. The modified virus is intended to make contact with a target Beta cell located in the Islets of Langerhans in the pancreas by means of the modified virus's exterior probes including one or more probes meant to engage GPR40 exterior cell-surface receptors on a Beta cell. Once the virus's exterior probes engage a target Beta cell's receptors, the modified virus inserts into the target cell one or more exogenous ribosomal ribonucleic acid molecules along with one or more exogenous messenger ribonucleic acid molecules it is carrying. A virus-like transport vehicle may be used in the place of a modified virus. Ribosomal RNA molecules connect to ribosome proteins and the resultant complex, referred to as a ribosome, surrounds the beginning segment of a mRNA molecule. Utilizing its own inherent coding, rRNA directs the ribosome pieces to build the ribosome complex around a particular strand of mRNA that the rRNA locates by matching the inherent coding the rRNA is carrying to a unique code on the mRNA. Medical disease states such as diabetes mellitus that are the result of a deficiency of one or more proteins can be successfully treated by utilizing viruses to insert the proper ribosomal RNA molecules along with the proper messenger RNA molecules into specific cells to enhance the production of proteins that are identified as being deficient, thus correcting the deficiency. The deficiency of insulin production is a prime example of a medical condition that is capable of being corrected by utilizing a modified virus to transport ribosomal RNA molecules along with messenger RNA molecules to assist ribosomes with producing the pro-insulin molecule, the insulin molecule, the insulin receptor molecule, prohormone convertase one (PC1), prohormone convertase two (PC2), and/or carboxypeptidase E. Delivering such ribosomal RNA molecules along with messenger RNA molecules to Beta cells for the purpose of enhancing the Beta cells' production of the insulin molecule and/or the insulin receptor offers a new and innovative treatment of diabetes and other protein deficient diseases.

### DETAILED DESCRIPTION

Diabetes mellitus is a medical condition often recognized when an individual's fasting blood glucose level is persistently higher than the generally accepted normal range of 60-110 mg/dl. An elevated blood glucose level may occur as the result of a lack of sufficient insulin; a lack of sufficient biologically effective insulin; a deficiency of the number of insulin receptors available to interact with insulin; a deficiency in the number of biologically active insulin receptors available to properly interact with insulin; insufficient release of insulin into the blood stream.

Insulin, a protein, is generated in Beta cells located in the Islets of Langerhans in the pancreas. Insulin is produced by decoding DNA through a process called transcription. Initially, transcription of the DNA produces a messenger ribonucleic acid (mRNA) molecule coded for the pro-insulin molecule. This mRNA coded for the ‘pro-insulin’ molecule, is then decoded by one or more ribosomes through a process called translation to produce a chain of amino acids that is referred to as the ‘pro-insulin’ molecule. The ‘pro-insulin’ molecule is modified by enzymes to produce the biologically active ‘insulin’ protein. Insulin molecules are stored in vacuoles in the Beta cells of the pancreas. Insulin is released from storage vacuoles in response to a rise in the level of glucose in the blood. Other proteins are manufactured in a similar fashion as pro-insulin and insulin.

Errors in the DNA or errors that occur in the process that generates the messenger RNA or a deficiency in the number of messenger RNA or a deficiency in the number of biologically active messenger RNA results in a deficiency of, or errors in the ‘pro-insulin’ molecule. Deficiencies in the biologically active enzymes intended to modify the ‘pro-insulin’ molecule to produce the biologically active insulin protein may result in deficiencies in adequate insulin production.

Correcting deficiencies or errors associated with the production of the protein insulin would correct diabetes mellitus, when diabetes mellitus is related to an insufficient quantity of biologically active insulin. Correcting deficiencies or errors associated with the production of insulin receptors would correct diabetes mellitus, when diabetes mellitus is related to an insufficient quantity of biologically active insulin receptors.

Messenger ribonucleic acids (mRNA) act as templates from which proteins are manufactured inside a cell. Ribosomal ribonucleic acids (rRNA) congregate with ribosomal proteins to produce a complex referred to as ribosome. Ribosomes attach to a mRNA and then in conjunction with charged tRNAs amino acid strings are constructed to form proteins.

The Hepatitis C virus (HCV) is comprised of an outer lipid bilayer envelope and an internal nucleocapsid. The virus's genetic payload is carried within the nucleocapsid. The HCV's naturally occurring genetic payload consists of a single molecule of linear positive sense, single stranded RNA approximately 9600 nucleotides in length, which includes: the structural proteins of C, E1, E2, the nonstructural proteins NS1, NS2, NS3, NS4A, NS4B, NS5A, NS5B, p7 and ARFP/F protein. Present on the surface of the outer envelope of the Hepatitis C virus are probes that detect receptors present on the surface of liver cells. The glycoproteins E1 and E2 have been identified to be affixed to the surface of HCV. Portions of the Hepatitis C virus genome, when separated into individual pieces, behave like messenger RNA. Naturally occurring HCV is constructed within a liver cell. Naturally occurring HCV is constructed with surface probes fashioned to recognize a receptor on the surface of a liver cell. Once the naturally occurring HCV's surface probe E2 engages a liver cell's CD81 receptor, and cofactors on the surface of HCV's exterior envelope engage the low density lipoprotein receptor (LDLR) or the scavenger receptor class B type I (SR-BI) on the liver cell, HCV then has the opportunity to insert its RNA genetic payload into the engaged target liver cell.

Replicating viruses and constructing viruses to carry DNA payloads is a form of manufacturing technology that has already been well established and is in use facilitating gene therapy. Replicating viruses and designing these viruses to carry ribosomal ribonucleic acid molecules along with one or more messenger ribonucleic acid molecules as the genetic payload would incorporate similar techniques as already proven useful in current gene therapy technologies.

To carry out the process to manufacture a modified medically therapeutic Hepatitis C virus, messenger RNA would be inserted into the host that would code for the general physical outer structures of the Hepatitis C virus. Messenger RNA would be inserted into the host that would generate surface probes that would target the surface receptors on Beta cells. Messenger RNA would be inserted into the host that would be used to generate copies of the therapeutic ribosomal RNA and therapeutic messenger RNA that would take the place of the Hepatitis C virus's innate genome. Therapeutic messenger RNA that would act as the modified HCV's genome would encode for proteins that would include the pro-insulin molecule, the insulin molecule, the insulin receptor, the enzyme prohormone convertase one, the enzyme prohormone convertase two, the enzyme carboxypeptidase E. Similar to how copies of a naturally occurring Hepatitis C virus are produced, assembled and released from a host cell, copies of the modified medically therapeutic Hepatitis C virus would be produced, assembled and released from a host cell.

To treat the various different forms of diabetes mellitus various combinations of messenger RNA would be inserted into the host, and the host would produce copies of modified Hepatitis C virus that target Beta cells and carry a genetic payload consisting of ribosomal RNA along with messenger RNA molecules that would consist of one or more copies of a ribosomal RNA along with a messenger RNA that codes for the insulin molecule, the insulin receptor, the enzyme prohormone convertase one, the enzyme prohormone convertase two, the enzyme carboxypeptidase E. Depending upon the physical size of the ribosomal RNA along with messenger RNAs and the available space inside the modified Hepatitis C virus, more than one type of ribosomal RNA or messenger RNA may be packaged inside a single modified Hepatitis C virus, which may produce more than one therapeutic action in a cell.

The modified Hepatitis C virus would be incapable of replication on its own due to the fact that the messenger RNA, that a naturally occurring Hepatitis C virus would normally carry, would not be present in the modified form of the Hepatitis C virus.

To treat diabetes, a quantity of modified Hepatitis C virus would be introduced into a patient's blood stream or tissues so that the modified form of virus could deliver the therapeutic genetic payload that it carries to Beta cells in the pancreas. When the probes on the surface of the modified Hepatitis C virus engage a cell-surface receptor or receptors on a Beta cell, the modified Hepatitis C virus will insert its therapeutic payload of ribosomal RNA molecules and messenger RNA molecules into the Beta cell to enhance the Beta cell's biologic function of producing insulin and/or insulin receptors. One such probe would be a probe located on the surface of the modified Hepatitis C virus that engages the exterior receptor GPR40, which appears to be an exterior receptor specific to Beta cells located in the Islets of Langerhans in the pancreas.

An alternative means to treat diabetes might include the use of a quantity of virus-like transport vehicles. The virus-like transport vehicles would be comprised of a bilayer lipid envelope and a nucleocapsid inner shell, similar to the construction of a virus, and such virus-like transport vehicles would carry a payload consisting of ribosomal RNA and messenger RNA molecules. Embedded in the virus-like transport vehicle's outer lipid bilayer surface envelope would be a quantity of probes that target cell-surface receptors on specific cells. The outer lipid bilayer acting as a matrix structure for the quantity of probes to be fixed into and extend out from the virus-like transport vehicle. The virus-like transport vehicles would be introduced into a patient's blood stream or tissues so that the virus-like transport vehicle could deliver the therapeutic genetic payload that it carries to Beta cells in the pancreas. When the probes on the surface of the virus-like transport vehicle engage a cell-surface receptor or receptors on a Beta cell located in the pancreas, the virus-like transport vehicle will insert its therapeutic payload of ribosomal RNA and messenger RNA into the Beta cell to enhance the Beta cell's biologic function of producing insulin and/or insulin receptors. One such probe could be a probe on the surface of the virus-like transport vehicle that engages the exterior receptor GPR40, which appears to be an exterior receptor specific to Beta cells located in the Islets of Langerhans in the pancreas. The ribosomal ribonucleic acid molecules carried as a payload in the virus-like transport vehicle would consist of a quantity of 5S ribosomal ribonucleic acid molecules, a quantity of 5.8S ribosomal ribonucleic acid molecules, a quantity of 12S ribosomal ribonucleic acid molecules, a quantity of 16S ribosomal ribonucleic acid molecules, a quantity of 18S ribosomal ribonucleic acid molecules, and/or a quantity of 28S ribosomal ribonucleic acid molecules. The quantity of each variety of ribosomal acid molecule determined by the need to decode messenger RNA molecules.

RNAs are generally degraded by enzymes known as ribonucleases or RNAases. Ribonucleases act to inactive the RNA molecules. Different RNAs are known to have different half-lives. The nucleotide sequencing of the rRNA molecule could be altered from that of the natural occurring molecule to lengthen or shorten the service half-life of the native rRNA. Depending upon the application, the rate of degradation of rRNA molecules by ribonucleases could be varied. By changing the nucleotide sequence of the rRNA molecule or by altering the folding characteristics of the rRNA molecule or by altering both the nucleotide sequence and the folding characteristics of the rRNA molecule, the rate of degradation of the rRNA molecules by ribonucleases could be caused to vary in length of time from seconds to minutes to hours to days to weeks to months to years. The rate of degradation of the rRNA molecules could be tailored to the treatment objectives of the rRNA molecule. Lengthening the service life of rRNA molecules such that the rRNAs could combine with ribosomal proteins to participate in ribosomes over a longer than the naturally occurring period of time would be especially useful in diabetic patients to facilitate a greater production of insulin molecules in the Beta cells and to increase the interval that would be required between treatments.

Messenger RNA molecules are comprised of three regions (or segments). These three regions include a (1) 5′ untranslatable region, (2) a coding region and (3) a 3′ untranslatable region. The ‘5′ untranslatable region’ acts as the initiation point for a ribosome to attach to the mRNA. The ‘coding region’ acts as the template from which a protein is constructed. An ‘untranslatable region’ represents a segment of a messenger RNA molecule that does not code for a protein and is not used to yield a protein and therefore ‘translation’ does not occur in such a region. The 3′ untranslatable region is associated with the degradation of the usefulness of the mRNA. Different mRNAs have different service life expectancies. The half-life of the naturally occurring mRNA that acts as the template responsible for the production of the protein ‘glucokinase’ is two hours. The half-life of the naturally occurring mRNA that produces the protein ‘alcohol dehydrogenase’ is ten hours. The half-life of the naturally occurring mRNA that produces the protein ‘glucuronidase’ is thirty hours. By modifying the nucleotides that comprise the 3′ untranslatable unit of an mRNA the service half-life of the mRNA may be altered to be lengthened or shortened depending upon the need for the quantity of protein and timeframe over which the mRNA is required to produce the protein coded in the mRNA's protein coding region.

Modifying the ‘coding region’ of a messenger RNA will modify the protein the messenger RNA will produce when the ribosomes decode such a modified messenger RNA. As demonstrated by the case of modifying the naturally occurring parathyroid hormone by administering a molecule that is comprised of fewer amino acids than the original PTH molecule to produce a beneficial therapeutic effect, modifying proteins the messenger RNAs produce will provide health care providers with an entirely new and widely spanning armamentarium of medically beneficial therapies.

The 5′ untranslatable region of a messenger RNA molecule is used to identify the messenger RNA and utilized as a point of attachment by ribosomes to the messenger RNA molecule. Modifying the 5′ untranslatable region by altering the nucleotide sequence in the 5′ untranslatable region may make it easier to identify a modified messenger ribonucleic acid molecules in a fashion that the modified ribonucleic acid molecules can be engaged by ribosomes. Altering the nucleotide sequence of the 5′ untranslatable region of a modified messenger ribonucleic acid molecule to create a unique identifier would facilitate ribosomes to preferentially engage the modified messenger ribonucleic acid molecule to preferentially produce the protein for which the modified messenger ribonucleic acid molecule is acting as a template. Supplying cells with exogenous rRNA molecules and exogenous mRNA molecules that are both coded with a similar unique identifier such that the rRNA molecules will engage the mRNA molecules, facilitates the production of a desired protein.

The act of altering the type of probe or probes present on the surface of the modified form of virus, the modified Hepatitis C virus, or the virus-like transport vehicle would change the target the modified form of virus or virus-like transport vehicle would seek. By changing the probes on the surface of the modified virus, the modified Hepatitis C virus, or the virus-like transport vehicle the payload carried by the modified virus, the modified Hepatitis C virus, or the virus-like transport vehicle could be delivered to any cell that carried on its surface a cell-surface receptor that would engage the probes the modified virus, the modified Hepatitis C virus, or the virus-like transport vehicle was carrying on its surface. In this fashion, specific payloads could be delivered to specific cells throughout the body.

A ‘messenger RNA’ carried by a modified form of virus, a modified Hepatitis C virus, or a virus-like transport vehicle may code or act as a template for ‘multiple proteins’. A messenger RNA may act as a template for multiple copies of the same protein. A messenger RNA may act as a template that will produce various different types of proteins. Utilizing naturally occurring proteases or exogenous proteases delivered with the messenger RNA or exogenous proteases delivered by a separate transport vehicle, the original protein produced by an exogenous messenger RNA molecule may be split into subunits, with each subunit representing a copy of the same protein or each subunit representing a different protein. Proteases carried by a modified form of virus, a modified Hepatitis C virus, or a virus-like transport vehicle are carried inside the outer shell of the respective vehicle, generally inside the nucleocapsid inner shell of the modified form of virus, a modified Hepatitis C virus, or a virus-like transport vehicle. By fitting multiple templates for a particular protein into a single exogenous messenger RNA and utilizing proteases to split the proteins generated by the exogenous messenger RNA at specific sites along the proteins, multiple copies of the same smaller protein subunit could be generated. By fitting multiple templates for different proteins into a single exogenous messenger RNA and utilizing proteases to split the proteins generated by this exogenous messenger RNA at specific sites along the proteins, one or more copies of different smaller protein subunits could be generated. The utilization of proteases will significantly increase the efficiency by which medically therapeutic proteins are generated inside a cell.

A messenger RNA carried by a modified form of virus, a modified Hepatitis C virus, or a virus-like transport vehicle may be comprised of multiple translatable messenger RNA subunits. Utilizing naturally occurring ribonucleases or exogenous ribonucleases delivered with the messenger RNA or exogenous ribonucleases delivered by a separate transport vehicle, the original exogenous messenger RNA molecule may be split into translatable subunits. The original exogenous messenger RNA molecule may be physically split or degraded into subunits representing templates, that by the process of translation, will produce copies of the same protein or the original exogenous messenger RNA molecule may be physically split or degraded into subunits representing templates, that by the process of translation, will produce copies of different proteins. Ribonucleases carried by a modified form of virus, a modified Hepatitis C virus, or a virus-like transport vehicle are carried inside the outer shell of the respective vehicle, generally inside the nucleocapsid inner shell of the modified form of virus, a modified Hepatitis C virus, or a virus-like transport vehicle. Ribonucleases carried by a modified form of virus, a modified Hepatitis C virus, or a virus-like transport vehicle may be required to be in primitive state or inactive state that does not cause degradation of the messenger RNA while the messenger RNA is being packaged into the vehicle during initial production, or cause degradation of the messenger RNA while the messenger RNA is being transported in a modified form of virus, a modified Hepatitis C virus, or a virus-like transport vehicle on its way to a host cell. The utilization of ribonucleases will significantly increase the efficiency by which medically therapeutic proteins are generated inside a cell.

By providing Beta cells with the above-mentioned ribosomal RNAs along with messenger RNAs, the capacity of Beta cells to carrying out the biologic processes of producing insulin and recognizing and responding to blood glucose levels is enhanced, which results in an efficient means to control the glucose levels in the blood stream on a constant and persistent basis utilizing innate regulatory mechanisms and thus diabetes mellitus can be effectively treated and the harmful effects of this disease can be averted.

By providing any target cell with medically therapeutic ribosomal RNAs along with medically therapeutic messenger RNAs, to enhance a cell's capacity to produce one or more proteins, any protein deficiency can be effectively treated and the harmful effects of the protein deficiency can be averted.

1. A medical treatment device comprising a modified form of a virus containing a quantity of medically therapeutic ribosomal ribonucleic acid molecules along with a quantity of medically therapeutic messenger ribonucleic acid molecules,
whereby said modified form of a virus is altered in a physical manner in relation to a naturally occurring virus,
whereby once said modified form of a virus inserts said quantity of ribosomal ribonucleic acid molecules into a biologically active cell, said quantity of ribosomal ribonucleic acid molecules are intended to engage ribosomal proteins to produce ribosomes, said ribosomes to decode said messenger ribosomal acid molecules to produce proteins, the result of which is intended to produce a beneficial medical therapeutic effect.

2. A medical treatment device comprising:
(a) a modified form of a virus carrying on its surface a quantity of probes that target cell-surface receptors on certain biologically active cells,
(b) said modified form of virus containing a quantity of ribosomal ribonucleic acid molecules along with a quantity of messenger ribonucleic acid molecules,
whereby said quantity of ribosomal ribonucleic acid molecules along with said messenger ribonucleic acid molecules are to be inserted into said biologically active cells, said ribosomal ribonucleic acid molecules capable of interacting with ribosomal protein molecules that assemble to construct a ribosome, purpose of said ribosome to decode said messenger ribonucleic acid molecules the result being to produce a specific protein inside said metabolically active cell for the purpose of carrying out a beneficial medical therapeutic effect.

3. The medical device in claim 2 wherein said messenger ribonucleic acid molecules are modified in the 3′ untranslatable region by altering the nucleotide sequence in said 3′ untranslatable region in a manner that will result in said messenger ribonucleic acid molecules resisting degradation by cellular enzymes without compromising the functionality of said messenger ribonucleic acid molecules,
whereby said modification of said 3′ untranslatable region of said messenger ribonucleic acid molecules is in a fashion that will result in extending the service half-life of said messenger ribonucleic acid molecules, compared to similar naturally occurring said messenger ribonucleic acid molecules, to allow additional time for said ribosomes to decode the information present on said messenger ribonucleic acid molecules to produce said proteins, which will enhance said protein production by said ribosomes to produce a beneficial medical therapeutic effect.

4. The medical device in claim 2 wherein said messenger ribonucleic acid molecules are modified in the 5′ untranslatable region by altering the nucleotide sequence in said 5′ untranslatable region in a manner that will result in identifying said messenger ribonucleic acid molecules in a fashion that facilitates said messenger ribonucleic acid molecules to be engaged by said ribosomes,
whereby said alteration of said 5′ untranslatable region of said messenger ribonucleic acid molecules occurs in a fashion to create an identifying code in said nucleotide sequence, said identifying code to facilitate said ribosomes to preferentially engage said messenger ribonucleic acid molecules.

5. The medical device in claim 2 wherein said messenger ribonucleic acid molecules have been modified in the coding region by changing the nucleotide sequence of said messenger ribonucleic acid molecules in said coding region in a manner that will result in altering the structure of said proteins said messenger ribonucleic acid molecules will produce once said messenger ribonucleic acid molecules undergo the process of translation by said ribosomes, the intended result to produce a beneficial medical therapeutic effect,
whereby said messenger ribonucleic acid molecules have been modified in said coding region by altering said nucleotide sequence of said messenger ribonucleic acid molecules in said coding region in a manner which will alter said structure of said protein said messenger ribonucleic acid molecules will produce compared to a naturally occurring messenger ribonucleic acid molecule naturally fashioned as a template to produce a similar protein or similar type of protein.

6. The medical device in claim 2 wherein said quantity of ribosomal ribonucleic acid molecules selected from the group consisting of a quantity of 5S ribosomal ribonucleic acid molecules, a quantity of 5.8S ribosomal ribonucleic acid molecules, a quantity of 18S ribosomal ribonucleic acid molecules, and a quantity of 28S ribosomal ribonucleic acid molecules,
whereby said quantity of ribosomal ribonucleic acid molecules is to be determined by the need of said ribosomal ribonucleic acid molecules to function as mitochondrial ribosomal ribonucleic acid molecules versus cytoplasmic ribosomal ribonucleic acid molecules,
whereby said quantity of ribosomal ribonucleic acid molecules is to be determined by the physical size of said ribosomal ribonucleic acid molecules and the physical capacity of said modified form of a virus to carry said quantity of ribosomal ribonucleic acid molecules along with said quantity of messenger ribonucleic acid molecules.

7. The medical device in claim 2 wherein said ribosomal ribonucleic acid molecules' nucleotide sequence is modified in a manner which results in said ribosomal ribonucleic acid molecules being capable of resisting degradation by cellular enzymes without compromising functionality of said ribosomal ribonucleic acid molecules,
whereby said alteration to said nucleotide sequence in said ribosomal ribonucleic acid molecules will result in an extension of the service half-life of altered said ribosomal ribonucleic acid molecules, compared to naturally occurring ribosomal ribonucleic acid molecules, to allow additional time for said ribosomes to decode the information present on said messenger ribonucleic acid molecules to produce additional said protein molecules.

8. The medical device in claim 2 wherein said modified form of virus carries a quantity of proteases within said modified form of virus,
whereby following said protein being produced by translation of said messenger ribonucleic acid, said quantity of proteases act on said protein at a quantity of specific sites along said protein to produce subunits of said protein,
whereby following said protein being produced by translation of said messenger ribonucleic acid, said quantity of proteases act on said protein at a quantity of specific sites along said protein to produce a quantity of similar subunits of said protein,
whereby following said protein being produced by translation of said messenger ribonucleic acid, said quantity of proteases act on said protein at a quantity of specific sites along said protein to produce a quantity of differing subunits of said protein.

9. The medical device in claim 2 wherein said modified form of virus carries a quantity of ribonucleases within said modified form of virus,
whereby said quantity of ribonucleases act on said messenger ribonucleic acid molecules at a quantity of specific sites along said messenger ribonucleic acid molecules to produce a quantity of translatable subunits of said messenger ribonucleic acid molecules with each said subunit of said messenger ribonucleic acid molecule capable of participating in the process of translation to produce a quantity of copies of similar proteins,
whereby said quantity of ribonucleases act on said messenger ribonucleic acid molecules at a quantity of specific sites along said messenger ribonucleic acid molecules to produce a quantity of translatable subunits of said messenger ribonucleic acid molecules with each subunit of said messenger ribonucleic acid molecule capable of participating in the process of translation to produce a quantity of copies of differing proteins.

10. A medical treatment device comprising:
(a) a modified Hepatitis C virus,
(b) said modified Hepatitis C virus having been modified to have a quantity of surface probes to recognize, then engage one or more exterior cell-surface receptors on the surface of specific biologically active cells,
(c) said modified Hepatitis C virus expressing said quantity of surface probes intended to facilitate inserting into said Beta cell said modified Hepatitis C virus's genome that said modified Hepatitis C virus carries,
(d) said modified Hepatitis C virus further modified to have its own innate positive sense ribonucleic acid genome replaced by a quantity of ribosomal ribonucleic acid molecules along with a quantity of messenger ribonucleic acid molecules,
whereby said quantity of ribosomal ribonucleic acid molecules along with said messenger ribonucleic acid molecules are to be inserted into said biologically active Beta cells, said ribosomal ribonucleic acid molecules capable of interacting with ribosomal protein molecules that assemble to construct a ribosome, purpose of said ribosome to decode said messenger ribonucleic acid molecules, the result being to produce a specific protein inside said metabolically active Beta cell for the purpose of carrying out a beneficial therapeutic medical treatment,
whereby said beneficial therapeutic medical treatment is intended to assist in the medical management the medical condition known as diabetes mellitus.

11. The medical device in claim 10 wherein said messenger ribonucleic acid molecules are modified in the 3′ untranslatable region by altering the nucleotide sequence in said 3′ untranslatable region in a manner that will result in said messenger ribonucleic acid molecules resisting degradation by cellular enzymes without compromising the functionality of said messenger ribonucleic acid molecules,
whereby said modification of said 3′ untranslatable region of said messenger ribonucleic acid molecules is in a fashion that will result in extending the service half-life of said messenger ribonucleic acid molecules, compared to similar naturally occurring said messenger ribonucleic acid molecules, to allow additional time for said ribosomes to decode the information present on said messenger ribonucleic acid molecules to produce said proteins, which will enhance said protein production by said ribosomes to produce a beneficial medical therapeutic effect.

12. The medical device in claim 10 wherein said messenger ribonucleic acid molecules are modified in the 5′ untranslatable region by altering the nucleotide sequence in said 5′ untranslatable region in a manner that will result in identifying said messenger ribonucleic acid molecules in a fashion that facilitates said messenger ribonucleic acid molecules to be engaged by said ribosomes,
whereby said alteration of said 5′ untranslatable region of said messenger ribonucleic acid molecules occurs in a fashion to create an identifying code in said nucleotide sequence, said identifying code facilitating said ribosomes to preferentially engage said messenger ribonucleic acid molecules.

13. The medical device in claim 10 wherein said messenger ribonucleic acid molecules have been modified in the coding region by changing the nucleotide sequence of said messenger ribonucleic acid molecules in said coding region in a manner that will result in altering the structure of said proteins said messenger ribonucleic acid molecules will produce once said messenger ribonucleic acid molecules undergo the process of translation by said ribosomes, the intended result to produce a beneficial medical therapeutic effect,
whereby said messenger ribonucleic acid molecules have been modified in said coding region by altering said nucleotide sequence of said messenger ribonucleic acid molecules in said coding region in a manner which will alter said structure of said protein said messenger ribonucleic acid molecules will produce compared to a naturally occurring messenger ribonucleic acid molecule naturally fashioned as a template to produce a similar protein or similar type of protein.

14. The medical device in claim 10 wherein said quantity of ribosomal ribonucleic acid molecules selected from the group consisting of a quantity of 5S ribosomal ribonucleic acid molecules, a quantity of 5.8S ribosomal ribonucleic acid molecules, a quantity of 18S ribosomal ribonucleic acid molecules, and a quantity of 28S ribosomal ribonucleic acid molecules,
whereby said quantity of ribosomal ribonucleic acid molecules is to be determined by the need of said ribosomal ribonucleic acid molecules to function as mitochondrial ribosomal ribonucleic acid molecules versus cytoplasmic ribosomal ribonucleic acid molecules,
whereby said quantity of ribosomal ribonucleic acid molecules is to be determined by the size of said ribosomal ribonucleic acid molecules and the physical capacity of said modified Hepatitis C virus to carry said quantity of ribosomal ribonucleic acid molecules.

15. The medical device in claim 10 wherein said ribosomal ribonucleic acid molecules' nucleotide sequence is modified in a manner which results in said ribosomal ribonucleic acid molecules being capable of resisting degradation by cellular enzymes without compromising functionality of said ribosomal ribonucleic acid molecules,
whereby said alteration to said nucleotide sequence in said ribosomal ribonucleic acid molecules will result in an extension of the service half-life of altered said ribosomal ribonucleic acid molecules, compared to naturally occurring ribosomal ribonucleic acid molecules, to allow additional time for said ribosomes to decode the information present on said messenger ribonucleic acid molecules to produce additional said protein molecules.

16. The medical device in claim 10 wherein a portion of said quantity of surface probes located on the exterior of said modified Hepatitis C virus are fashioned to recognize and engage GPR40 exterior cell-surface receptors on said Beta cell located in said Islets of Langerhans in said pancreas.

17. The medical device in claim 10 wherein said modified Hepatitis C virus carries a quantity of proteases within said modified Hepatitis C virus,
whereby following said protein being produced by translation of said messenger ribonucleic acid, said quantity of proteases act on said protein at a quantity of specific sites along said protein to produce subunits of said protein,
whereby following said protein being produced by translation of said messenger ribonucleic acid, said quantity of proteases act on said protein at a quantity of specific sites along said protein to produce a quantity of similar subunits of said protein,
whereby following said protein being produced by translation of said messenger ribonucleic acid, said quantity of proteases act on said protein at a quantity of specific sites along said protein to produce a quantity of differing subunits of said protein.

18. The medical device in claim 10 wherein said modified Hepatitis C virus carries a quantity of ribonucleases within said modified Hepatitis C virus,
whereby said quantity of ribonucleases act on said messenger ribonucleic acid molecules at a quantity of specific sites along said messenger ribonucleic acid molecules to produce a quantity of translatable subunits of said messenger ribonucleic acid molecules, with each said subunit of said messenger ribonucleic acid molecules capable of participating in the process of translation to produce a quantity of copies of similar proteins,
whereby said quantity of ribonucleases act on said messenger ribonucleic acid molecules at a quantity of specific sites along said messenger ribonucleic acid molecules to produce a quantity of translatable subunits of said messenger ribonucleic acid molecules, with each said subunit of said messenger ribonucleic acid molecules capable of participating in the process of translation to produce a quantity of copies of differing proteins.

19.-27. (canceled)
